(19) United States
(12) Patent Application Publication
(10) Pub. No.: US 2018/0040433 A1
(43) Pub. Date: Feb. 8, 2018

(54) FOOT POWERED SURGICAL DEVICE

(71) Applicant: COVIDIEN LP, Mansfield, MA (US)

(72) Inventor: Tejas Inamdar, Boston, MA (US)

(21) Appl. No.: 15/666,018

(22) Filed: Aug. 1, 2017

Related U.S. Application Data
(60) Provisional application No. 62/370,546, filed on Aug. 3, 2016.

(51) Int. Cl. H01H 3/14; H03K 17/96; A61B 18/00; A61B 17/32

(52) U.S. Cl. CPC H01H 3/14; A61B 17/320068; H03K 17/96; A61B 18/00; A61B 17/32002; A61B 2017/00973

(57) ABSTRACT

Surgical systems include a handheld surgical device having an end effector and a drive assembly configured to drive the end effector, a foot pedal configured to receive input mechanical energy from a user and to output mechanical energy, and a converter. In systems, the drive assembly is an electrically-powered drive assembly and converter is configured to receive the output mechanical energy from the foot pedal and to output electrical energy to the electrically-powered drive assembly. In other systems, the drive assembly is mechanically-powered and the converter is configured to receive the output mechanical energy from the foot pedal and to output a different mechanical energy to the mechanically-powered drive assembly.

### CROSS-REFERENCE TO RELATED APPLICATIONS

This application claims the benefit of U.S. Provisional Patent Application No. 62/370,546, filed on Aug. 3, 2016, the entire contents of which are hereby incorporated herein by reference.

### BACKGROUND

Handheld surgical devices may be powered by activation of a button on a handset of the surgical device or by activation of a foot pedal connected to the surgical device. As such, the surgical device may be powered on when the button or foot pedal is activated, and powered off when the button or foot pedal is released.

### SUMMARY

As used herein, the term “distal” refers to the portion that is being described that is further from a user, while the term “proximal” refers to the portion that is being described that is closer to a user. Further, to the extent consistent, any of the aspects described herein may be used in conjunction with any of the other aspects described herein.

Provided in accordance with aspects of the present disclosure is a surgical system including a handheld surgical device, a foot pedal, and a converter. The handheld surgical device includes an end effector and an electrically-powered drive assembly configured to drive the end effector assembly. The foot pedal is configured to receive input mechanical energy from a user and to output mechanical energy. The converter is operably coupled to the handheld surgical device and the foot pedal and is configured to receive the output mechanical energy from the foot pedal and to output electrical energy to the electrically-powered drive assembly.

In an aspect of the present disclosure, a storage device is operably coupled to the converter and configured to receive at least some of the output electrical energy from the converter.

In another aspect of the present disclosure, an electrically-powered auxiliary device is operably coupled to the handheld surgical device and configured to be powered by the output electrical energy from the converter.

In another aspect of the present disclosure, the input mechanical energy is rotational motion and the output mechanical energy is rotational motion. Alternatively, the input mechanical energy is longitudinal motion and wherein the output mechanical energy is rotational motion. Alternatively, the input mechanical energy is rotational motion and wherein the output mechanical energy is longitudinal motion.

In still another aspect of the present disclosure, the foot pedal is coupled to the converter via a connector including mechanical energy-transmission components disposed therein.

In yet another aspect of the present disclosure, the converter is coupled to the handheld surgical device via a connector including one or more electrical wires disposed therein.

In still yet another aspect of the present disclosure, the foot pedal includes a hinged platform pivotable between an actuated position and an un-actuated position. Alternatively, the foot pedal includes a wheel and at least one pedal coupled to the wheel for rotating the wheel about an axis. Alternatively, the foot pedal includes a slider slidable between a first position and a second position.

Another surgical system provided in accordance with aspects of the present disclosure includes a handheld surgical device, a foot pedal, and a converter. The handheld surgical device includes an end effector and a drive assembly configured to drive the end effector assembly. The foot pedal is configured to receive input mechanical energy from a user and to output mechanical energy. The converter is operably coupled to the handheld surgical device and the foot pedal and configured to receive the output mechanical energy from the foot pedal and to output a different mechanical energy to the drive assembly.

In an aspect of the present disclosure, the converter is configured to receive rotational motion as the output mechanical energy and output longitudinal motion as the different mechanical energy. Alternatively, the converter is configured to receive longitudinal motion as the output mechanical energy and output rotational motion as the different mechanical energy.

In another aspect of the present disclosure, the different mechanical energy is mechanical motion of a different speed as compared to a speed of mechanical motion of the output mechanical energy.

In yet another aspect of the present disclosure, an auxiliary device is operably coupled to the handheld surgical device and configured to be powered by the different mechanical energy from the converter.

In still another aspect of the present disclosure the foot pedal is coupled to the converter via a first connector including first mechanical energy-transmission components. Additionally or alternatively, the converter is coupled to the drive assembly via a second connector including second mechanical energy-transmission components.

In still yet another aspect of the present disclosure, the foot pedal includes a hinged platform pivotable between an actuated position and an un-actuated position. Alternatively, the foot pedal includes a wheel and at least one pedal coupled to the wheel for rotating the wheel about an axis. Alternatively, the foot pedal includes a slider slidable between a first position and a second position.

### BRIEF DESCRIPTION OF THE DRAWINGS

The above and other aspects and features of the present disclosure will become more apparent in view of the following detailed description when taken in conjunction with the accompanying drawings, in which:

FIG. 1A is a block diagram of a surgical system provided in accordance with aspects of the present disclosure;

FIG. 1B is a side view of another surgical system provided in accordance with aspects of the present disclosure;

FIGS. 2A-2C are schematic illustrations of various foot pedal configurations for use with the surgical systems of FIGS. 1A and 1B;

FIGS. 3A-3D are schematic illustrations of various other foot pedal configurations for use with the surgical systems of FIGS. 1A and 1B;

FIG. 4 is a schematic illustration of still another foot pedal configuration for use with the surgical systems of FIGS. 1A and 1B; and

FIG. 5 is a schematic illustration of yet another foot pedal configuration for use with the surgical systems of FIGS. 1A and 1B.

### DETAILED DESCRIPTION

Handheld surgical devices such as resection devices may be hard to grip and actuate. The forces that a user can supply and the duration for which the user can supply that force to the device may be inadequate to successfully perform the procedure. Some handheld surgical devices may use a foot-activated electrical power switch that activates a motor in the handheld device when the switch is depressed and deactivates the motor when switch is released. These devices may supply more or less electrical power to the motor in handheld surgical device depending upon the corresponding compression of the foot pedal, but may require significant capital investment.

The present disclosure employs a foot pedal and a handheld surgical device and, in embodiments, a converter and/or storage device. Power, energy, and/or force used to operate the handheld device is/are provided directly or indirectly from the foot pedal. The handheld surgical device may also be configured to couple to other auxiliary devices such as fluid management systems as needed for various surgical procedures. These and other aspects and features of the present disclosure are detailed below.

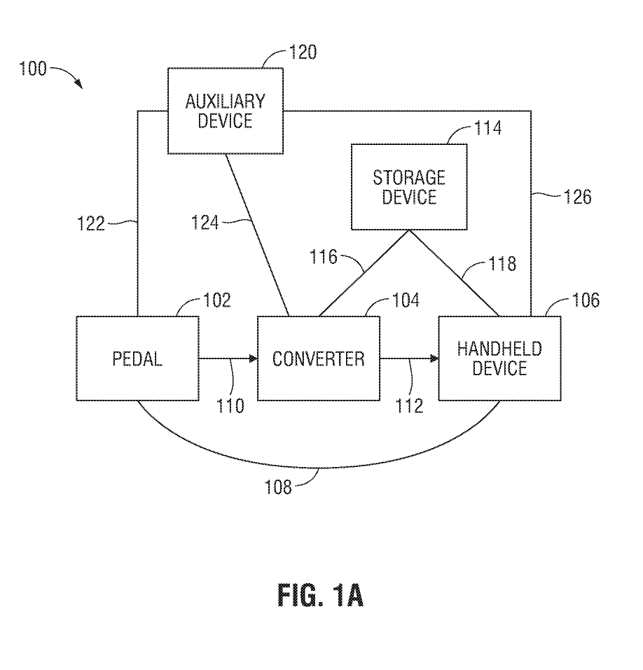

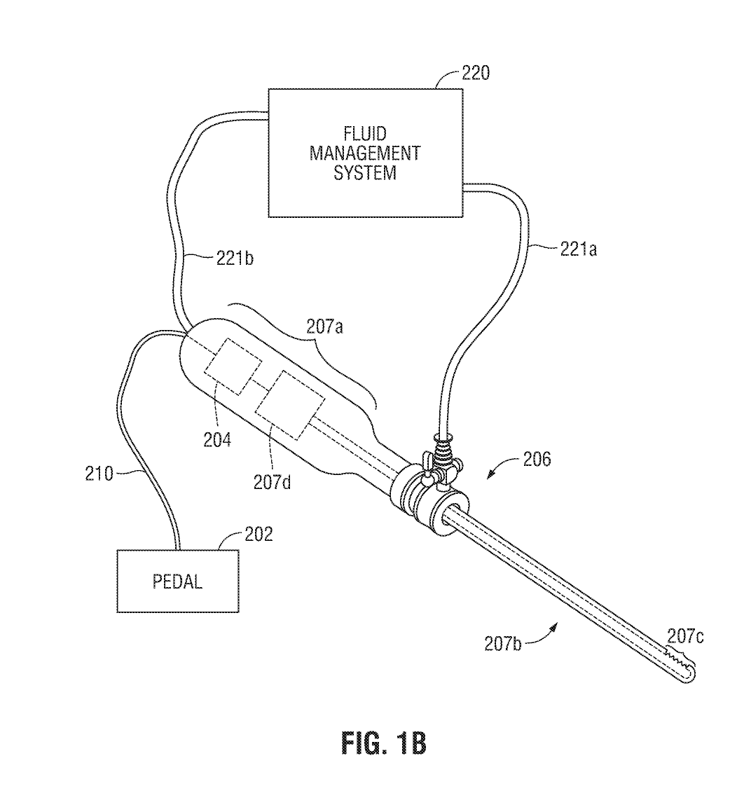

FIG. 1A is a block diagram of a surgical system 100 including a foot pedal 102 in communication with a handheld surgical device 106. Foot pedal 102 may be configured similarly to any of the embodiments of foot pedals detailed hereinbelow, or in any other suitable manner. In embodiments, foot pedal 102, when activated by a user, provides mechanical energy directly to handheld surgical device 106 via a connector 108. Alternatively or additionally, foot pedal 102, when activated, provides mechanical energy to a converter 104 via a connector 110. Converter 104, in turn, provides energy in some form, e.g., mechanical or electrical, to handheld surgical device 106 via a connector 112 and/or to a storage device 114, e.g., a battery or capacitor, via a connector 116. Storage device 114 may provide energy to handheld surgical device 106 via a connector 118, as required, or may provide the energy back to converter 104, via connector 112, for delivery to handheld surgical device 106. Converter 104 and/or storage device 114 may be contained within or on handheld surgical device 106, or may be separate therefrom. Converter 104 and/or storage device 114 may alternatively be contained within or on foot pedal 102.

Foot pedal 102 may additionally or alternatively, when activated, provide mechanical energy to one or more auxiliary devices 120, e.g., a fluid management system. More specifically, the mechanical energy may be provided from foot pedal 102 directly to handheld surgical device 106 or to converter 104 which in turn, provides energy to handheld surgical device 106 for powering (mechanically and/or electrically) auxiliary device(s) 120. Alternatively, the mechanical energy from foot pedal 102 may be provided to auxiliary device(s) 120 directly via one or more connectors 122 or through converter 104 via one or more connectors 124. In either of these configurations, auxiliary device(s) 120 is connected to handheld surgical device 106 via one or more connectors 126, e.g., to provide fluid inflow and outflow capabilities or other auxiliary function(s).

Referring still to FIG. 1A, the connectors transferring mechanical energy from foot pedal 102, e.g., connector 108, connector 110, and connector 122, may include one or more torsion cables, chains, belts, and/or other suitable connectors capable of transferring mechanical energy.

In embodiments, converter 104 may be configured to adjust the mechanical energy provided thereto, e.g., by adjusting the speed, torque, and/or type of mechanical energy (uni-directional linear motion, reciprocating linear motion, rotational motion, combined rotational and linear motion, etc.), and output the adjusted mechanical energy via connector(s) 112, 116, and/or 124. In such configurations, converter 104 may implement adjustable gear ratios, clutches, and/or other features to adjust the mechanical energy to meet operating parameters for the particular handheld surgical device 106 to be used. The connectors transferring the adjusted mechanical energy from converter 104, e.g., connector 112, connector 116, and connector 124, may include one or more torsion cables, chains, belts, and/or other suitable connectors capable of transferring mechanical energy.

In embodiments, convertor 104 is additionally or alternatively configured to store mechanical energy within storage device 114 (which may be part of or separate from converter 104). This may be accomplished, for example, by loading a spring or spinning a flywheel. Thus, the user can build up stored mechanical energy within storage device 114 by operation of foot pedal 102, enabling the stored mechanical energy to be extracted as needed from convertor 104 to operate handheld surgical device 106. In such configurations, the connectors to/from storage device 114, e.g., connector 116 and/or connector 118 may include one or more torsion cables, chains, belts, and/or other suitable connectors capable of transferring mechanical energy.

With continued reference to FIG. 1A, in embodiments, converter 104 may receive mechanical energy (uni-directional linear motion, reciprocating linear motion, rotational motion, combined rotational and linear motion, etc.) from foot pedal 102 and convert the mechanical energy into electrical energy, e.g., for output to handheld surgical device 106, storage device 114, and/or auxiliary device(s) 120. That is, in such configurations, converter 104 functions as a transducer. In such configurations, storage device 114 may be, for example, a battery or capacitor; handheld surgical device 106 and/or auxiliary device 120 may include electric motors or other electric-powered drives or outputs; and/or the connectors downstream of converter 104, e.g., connector 112, connector 116, connector 118, connector 124, and/or connector 126, for example, may include one or more electrical wires configured to transmit electrical energy therealong. Converter 104 may be configured to regulate the electrical power supplied to handheld surgical device 106 and/or auxiliary device(s) 120 independent of the speed of and/or pressure applied to foot pedal 102, or may output the electrical power in proportion to the speed of and/or pressure applied to foot pedal 102.

As illustrated in FIG. 1B, in embodiments, a surgical system 200 is provided wherein the handheld surgical device is a resection tool 206 and is coupled to a foot pedal 202 by way of a connector 210 and coupled to a fluid management system 220 by way of connectors 221a, 221b. Resection tool 206 generally includes a handle portion 207a, an elongated body portion 207b extending distally from the handle portion 207a, and an end effector assembly 207c, e.g., a reciprocating cutter, a rotational cutter, or a combination reciprocating and rotating cutting, extending distally from elongated body portion 207b. Resection tool 206 may house converter 204 within handle portion 207a thereof (as shown), or converter 204 may be separate therefrom.

In embodiments where resection tool 206 is electrically powered, resection tool 206 may include a drive assembly 207d including a motor electrically coupled to converter 204 and mechanically coupled to end effector assembly 207c to drive movement of end effector assembly 207c upon activation. The storage device (not shown), in embodiments where provided, may be disposed within handle portion 207a, or may be separate therefrom, for storing electrical energy for later delivery to drive assembly 207d.

In embodiments where resection tool 206 is mechanically powered, converter 204, if so provided, adjusts the mechanical energy received from foot pedal 202 and provides an appropriate output to drive assembly 207d and/or the storage device (not shown), in embodiments where such is provided. Alternatively, converter 204 and drive assembly 207d may be integrated with one another. In either configuration, drive assembly 207d may include, for example, gears, pulleys, cam structures, drive screws, cables, chains, belts, and/or other suitable drive structures to effect operation of end effector assembly 207c in response to activation of drive assembly 207d.

Fluid management system 220 includes one or more fluid pumps, fluid supply reservoirs, and/or fluid collection reservoirs, and includes an inflow connector 221a and an outflow connector 221b to respectively permit fluid inflow into and fluid outflow from a surgical site. Fluid management system 220 may be electrically powered by converter 204 or, if provided, by the storage device (not shown), or mechanically powered directly by foot pedal 202, by converter 204, if provided, by the storage device (not shown), or by drive assembly 207d.

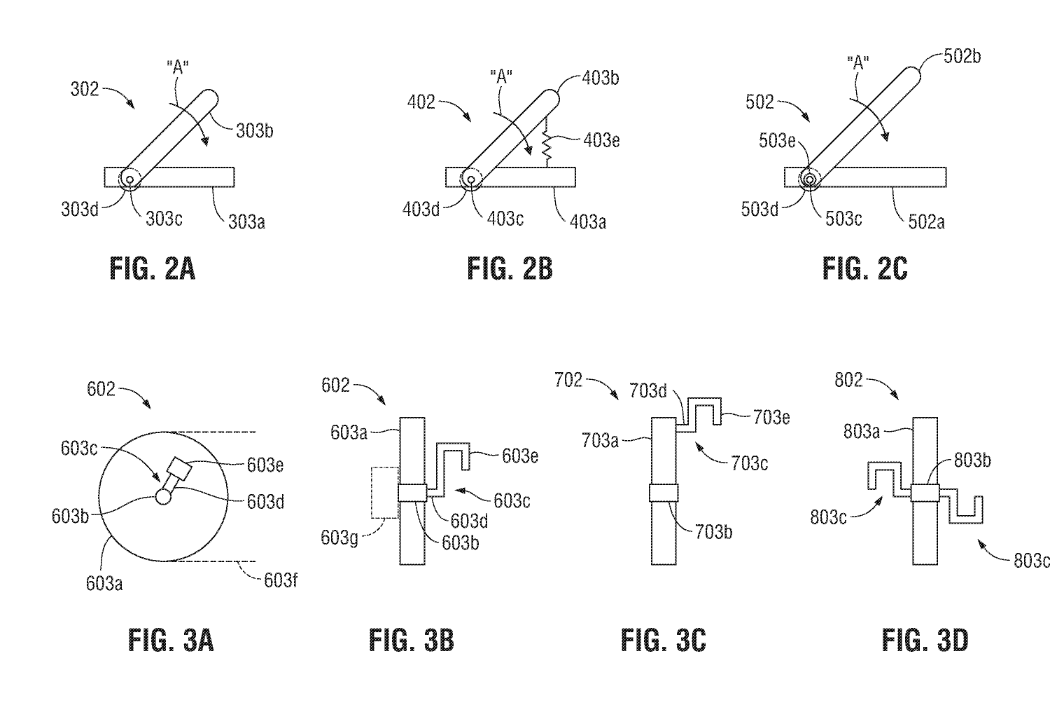

Turning now to FIGS. 2A-5, a variety of foot pedal configurations for use with system 100 (FIG. 1A), system 200 (FIG. 1B), or any other suitable surgical system are provided in accordance with the present disclosure and detailed below. Such foot pedal configurations includes treadle action configurations, pedal configurations similar to a bicycle, pedals configured to convert rotational motion into longitudinal motion, pedals configured to convert longitudinal motion into rotational motion, etc.

FIGS. 2A-2C illustrate embodiments of treadle configuration foot pedals 302-502, respectively. For example, FIG. 2A illustrates a foot pedal 302 including a base 303a and a hinged platform 303b pivotably coupled to base 303a via a pivot pin 303c towards an end of hinged platform 303b such that hinged platform 303b defines a cantilever configuration. Hinged platform 303b is selectively depressible relative to base 303a to rotate pivot pin 303c relative to base 303a. More specifically, hinged platform 303b is movable through a radiused arc “A” about pivot pin 303c and relative to base 303a between an un-actuated position, wherein the free end of hinged platform 303b is farther spaced-apart from base 303a, and an actuated position, wherein the free end of hinged platform 303b is closer to base 303a. Actuation of hinged platform 303b rotates pivot pin 303c relative to base 303a. As such, pivot pin 303c may be coupled to an output device for outputting rotational motion thereto, e.g., for direct or ultimate delivery to a surgical device, auxiliary device, storage device, etc. Foot pedal 302 may include a one-way mechanism 303d configured such that rotational motion is imparted from pivot pin 303c in only one direction, e.g., in the actuation direction of hinged platform 303a. One-way mechanism 303d may include a clutch, pawl/ratchet mechanism, etc. Other output configurations are also contemplated.

FIGS. 2B and 2C illustrates foot pedals 402 and 502, respectively, similar to foot pedal 302 (FIG. 2A) and each including a base 403a, 503a and a hinged platform 403b, 503b pivotably coupled to base 403a, 503a via a pivot pin 403c, 503c towards an end of hinged platform 403b, 503b and selectively depressible relative to base 303a to move hinged platform 403b, 503b through a radiused arc “A” about pivot pin 403c, 503c, thus rotating pivot pin 403c, 503c relative to base 403a, 503a, respectively. Foot pedals 402, 502 move between an un-actuated position and an actuated position, similarly as foot pedal 302 (FIG. 2A), to output rotational motion from pivot pin 403c, 503c, e.g., for direct or ultimate delivery to a surgical device, auxiliary device, storage device, etc. Foot pedals 402, 502 may also include one-way mechanisms 403d, 503d. Foot pedals 402, 502 differ from foot pedal 302 (FIG. 2A) in that foot pedals 402, 502 include biasing members 403e, 503e configured to bias hinged platforms 403b, 503b towards the un-actuated position. Biasing member 403e of foot pedal 402 is a compression spring extending between hinged platform 403b, towards the free end thereof, and base 403a. Biasing member 503e of foot pedal 502 is a torsion spring disposed about pivot pin 503c. Other suitable biasing members are also contemplated.

FIGS. 3A-3D illustrate foot pedal configurations similar to a bicycle. FIGS. 3A and 3B, for example, illustrate a foot pedal 602 including a wheel 603a disposed about a central pivot pin 603b, and a pedal 603c coupled to central pivot pin 603b. Pedal 603c includes a base 603d coupled to central pivot pin 603b, and a lever 603e, configured to receive a foot of a user, extending from base 603d. As a result of this configuration, urging of lever 603e to rotate about the axis of central pivot pin 603b rotates pedal 603c, central pivot pin 603b, and wheel 603a about the axis of central pivot pin 603b. As illustrated in FIG. 3A, wheel 603a may include an output mechanism 603f, e.g., a belt, disposed thereabout for receiving the rotational motion from wheel 603a. Alternatively or additionally, as illustrated in FIG. 3B, central pivot pin 603b may be coupled to an output mechanism 603g, e.g., a gear box, for receiving the rotational motion from central pivot pin 603b. Other suitable output configurations are also contemplated.

FIG. 3C illustrates a foot pedal 702 similar to foot pedal 602 (FIGS. 3A and 3B) and including a wheel 703a disposed about a central pivot pin 703b, and a pedal 703c coupled to wheel 703a towards the outer annular periphery thereof. Pedal 703c includes a base 703d coupled to wheel 703a and a lever 703e, configured to receive a foot of a user, extending from base 703d. Urging of lever 703e to rotate about the axis of central pivot pin 703b rotates pedal 703c, central pivot pin 703b, and wheel 703a about the axis of central pivot pin 703b. Foot pedal 702 may be coupled to any suitable output for outputting rotational motion thereto.

FIG. 3D illustrates a foot pedal 802 similar to foot pedal 602 (FIGS. 3A and 3B) and including a wheel 803a disposed about a central pivot pin 803b. Foot pedal 802 differs from foot pedal 602 (FIGS. 3A and 3B) in that, rather than providing a single pedal 603c (FIGS. 3A and 3B), foot pedal 802 includes a pair of pedals 803c, one disposed on each side of wheel 803a. Thus, foot pedal 802 enables two-footed actuation. Foot pedal 802 may otherwise be similar to and/or include any of the features of foot pedal 602 (FIGS. 3A and 3B).

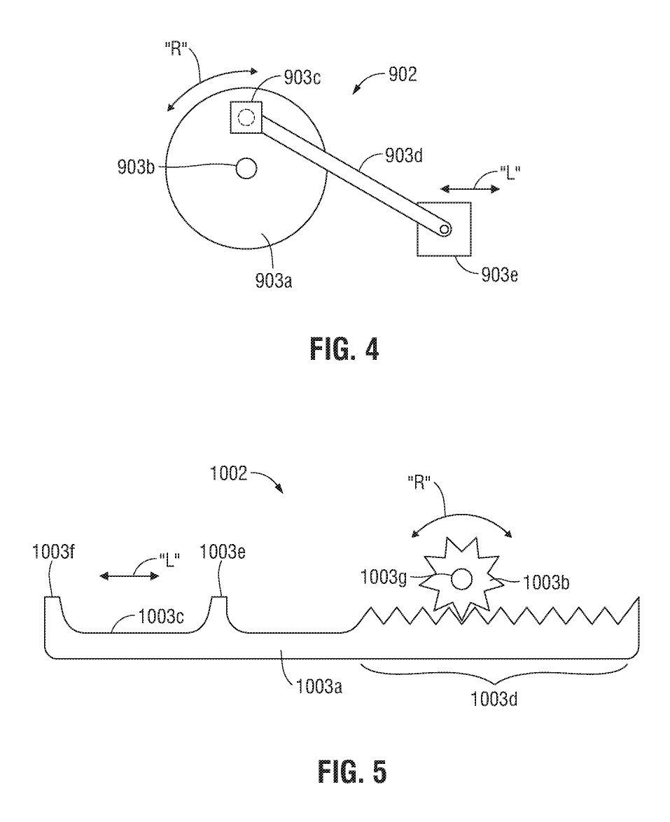

Turning to FIG. 4, another foot pedal provided in accordance with the present disclosure is shown as foot pedal 902. Foot pedal 902 is configured to convert rotational motion into longitudinal motion and includes a wheel 903a, e.g., a standard wheel or a flywheel, disposed about a central pivot pin 903b, and a pedal 903c coupled to wheel 903a towards the outer annular periphery thereof, a linkage 903d, and an output member 903e. Linkage 903d is pivotably coupled to pedal 903c at one end portion thereof and pivotably coupled to output member 903e, e.g., a gearbox, pulley system, etc., at the opposite end portion thereof. As a result of this configuration, urging of pedal 903c to rotate about the axis of central pivot pin 903b rotates pedal 903c to, in turn, rotate wheel 903a about the axis of central pivot pin 903b, thereby pushing or pulling linkage 903d (depending upon the position of the pedal 903c) to, in turn, longitudinally translate output member 903e. In this manner, rotational input provided to foot pedal 902, as indicated by arrows “R,” is converted into longitudinal output from foot pedal 902, as indicated by arrows “L.” Foot pedal 902 may be configured to output a reciprocating longitudinal motion or may include a one-way mechanism to only output longitudinal motion in a single direction.

In embodiments where wheel 903a is a flywheel, the energy stored therein helps regulate the output speed and to keep the crank in motion. The configuration, e.g., size and weight, of linkage 903d may be selected so as to influence the amount of energy stored and the rotational speed of the flywheel.

With reference to FIG. 5, another foot pedal provided in accordance with the present disclosure is shown as foot pedal 1002. Foot pedal 1002 is configured to convert longitudinal motion into rotational motion and includes a slider 1003a and a pinion 1003b. Slider 1003a includes a first end portion defining a foot-receiving socket 1003c and a second end portion defining a rack 1003d. Foot-receiving socket 1003c is configured to receive the foot of a user and includes front and rear stops 1003e, 1003f configured to inhibit longitudinal motion of the user's foot relative to slider 1003a when disposed within socket 1003c. To this end, front stop 1003e and/or rear stop 1003f may be adjustable to enable secure receipt of a user's foot within socket 1003c regardless of the user's foot size.

Rack 1003d of slider 1003a is disposed in meshed engagement with pinion 1003c. As a result of this configuration, urging of socket 1003c of slider 1003a to move longitudinally by a user's foot disposed therein urges rack 1003d of slider 1003a to move longitudinally, thereby rotating pinion 1003c about the axis of pivot pin 1003g, which supports pinion 1003c thereon. Thus, longitudinal input provided to foot pedal 1002, as indicated by arrows “L,” is converted into rotational output from foot pedal 1002, as indicated by arrows “R.” Foot pedal 1002 may be configured to output rotational motion in two directions or may include a one-way mechanism to only output rotational motion in a single direction. With respect to the output of foot pedal 1002, pivot pin 1003g may be coupled to an output device to impart rotational output thereto and/or an output device, e.g., a gearbox, may be operably coupled to pinion 1003c to receive rotational output therefrom.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A surgical system, comprising:
a handheld surgical device including an end effector and an electrically-powered drive assembly configured to drive the end effector assembly;
a foot pedal configured to receive input mechanical energy from a user and to output mechanical energy; and
a converter operably coupled to the handheld surgical device and the foot pedal, the converter configured to receive the output mechanical energy from the foot pedal and to output electrical energy to the electrically-powered drive assembly.

2. The surgical system according to claim 1, further comprising a storage device operably coupled to the converter, the storage device configured to receive at least some of the output electrical energy from the converter.

3. The surgical system according to claim 1, further comprising an electrically-powered auxiliary device operably coupled to the handheld surgical device, wherein the electrically-powered auxiliary device is powered by the output electrical energy from the converter.

4. The surgical system according to claim 1, wherein the input mechanical energy is rotational motion and wherein the output mechanical energy is rotational motion.

5. The surgical system according to claim 1, wherein the input mechanical energy is longitudinal motion and wherein the output mechanical energy is rotational motion.

6. The surgical system according to claim 1, wherein the input mechanical energy is rotational motion and wherein the output mechanical energy is longitudinal motion.

7. The surgical system according to claim 1, wherein the foot pedal is coupled to the converter via a connector including mechanical energy-transmission components disposed therein.

8. The surgical system according to claim 1, wherein the converter is coupled to the handheld surgical device via a connector including one or more electrical wires disposed therein.

9. The surgical system according to claim 1, wherein the foot pedal includes a hinged platform pivotable between an actuated position and an un-actuated position.

10. The surgical system according to claim 1, wherein the foot pedal includes a wheel and at least one pedal coupled to the wheel, the at least one pedal rotatable about an axis to thereby rotate the wheel about the axis.

11. The surgical system according to claim 1, wherein the foot pedal includes a slider slidable between a first position and a second position.

12. A surgical system, comprising:
a handheld surgical device including an end effector and a drive assembly configured to drive the end effector assembly;
a foot pedal configured to receive input mechanical energy from a user and to output mechanical energy; and
a converter operably coupled to the handheld surgical device and the foot pedal, the converter configured to receive the output mechanical energy from the foot pedal and to output a different mechanical energy to the drive assembly.

13. The surgical system according to claim 12, wherein the converter is configured to receive rotational motion as the output mechanical energy and output longitudinal motion as the different mechanical energy.

14. The surgical system according to claim 12, wherein the converter is configured to receive longitudinal motion as the output mechanical energy and output rotational motion as the different mechanical energy.

15. The surgical system according to claim 12, wherein the different mechanical energy is mechanical motion of a different speed as compared to a speed of mechanical motion of the output mechanical energy.

16. The surgical system according to claim 12, further comprising an auxiliary device operably coupled to the handheld surgical device, wherein the auxiliary device is powered by the different mechanical energy from the converter.

17. The surgical system according to claim 12, wherein at least one of: the foot pedal is coupled to the converter via a first connector including first mechanical energy-transmission components or the converter is coupled to the drive assembly via a second connector including second mechanical energy-transmission components.

18. The surgical system according to claim 12, wherein the foot pedal includes a hinged platform pivotable between an actuated position and an un-actuated position.

19. The surgical system according to claim 12, wherein the foot pedal includes a wheel and at least one pedal coupled to the wheel, the at least one pedal rotatable about an axis to thereby rotate the wheel about the axis.

20. The surgical system according to claim 12, wherein the foot pedal includes a slider slidable between a first position and a second position.
